(19) United States
(12) Patent Application Publication
(10) Pub. No.: US 2015/0238233 A1
(43) Pub. Date: Aug. 27, 2015

(54) Intervertebral Plate Systems and Methods of Use

(71) Applicant: Trinity Orthopedics, LLC, San Diego, CA (US)

(72) Inventors: James F. Marino, La Jolla, CA (US); Jamil Elbanna, San Diego, CA (US)

(21) Appl. No.: 14/419,916

(22) PCT Filed: Aug. 9, 2013
(86) PCT No.: PCT/US2013/054393

§ 371 Date: Feb. 5, 2015

Related U.S. Application Data
(60) Provisional application No. 61/681,521, filed on Aug. 9, 2012.

(51) Int. Cl. A61B 17/70; A61B 17/80

(52) U.S. Cl. CPC A61B 17/7059; A61B 17/8061; A61B 17/8033

(57) ABSTRACT

Provided are systems related to stabilizing adjacent superior and inferior vertebrae separated by a disc space that include a plate having at least one plate aperture and at least one bone screw sized and shaped to be positioned through the at least one plate aperture. The plate has a first cross-sectional area and thickness near a midline of the plate that is aligned with the intervertebral disc space upon deployment of the system, a second cross-sectional area and thickness located near a superior margin of the plate that is aligned with the superior vertebra, and a third cross-sectional area and thickness located near an inferior margin of the plate that is aligned with the inferior vertebra. The first cross-sectional area and thickness is greater than the second and third cross-sectional areas such that the plate projects in a fusiform manner both toward and away from the intervertebral disc space.

### REFERENCE TO PRIORITY DOCUMENTS

This application claims the benefit of priority under 35 U.S.C. §119(e) of co-pending U.S. Provisional Patent Application Ser. No. 61/681,521, filed Aug. 9, 2012. Priority of the aforementioned filing date is hereby claimed and the disclosure of the provisional patent application hereby incorporated by reference in its entirety.

### BACKGROUND

Immobilization of the spine is a surgical objective for achieving spinal fusion. Spine surgeons utilize various methods and implants to immobilize the spine in an effort to join one vertebra to another. These methods include the utilization of a plate and screws that bridge the gap between vertebrae or intervertebral disc space. There are a number of surgical plates available for this purpose in the lumbar, thoracic, and cervical spine.

### SUMMARY

In one aspect, provided are systems for stabilizing adjacent superior and inferior vertebrae separated by an intervertebral disc space. The system includes a plate having at least one plate aperture and at least one bone screw sized and shaped to be positioned through the at least one plate aperture. The plate has a first cross-sectional area and thickness near a midline of the plate that is aligned with the intervertebral disc space upon deployment of the system, a second cross-sectional area and thickness located near a superior margin of the plate that is aligned with the superior vertebra upon deployment of the system, and a third cross-sectional area and thickness located near an inferior margin of the plate that is aligned with the inferior vertebra upon deployment of the system. The first cross-sectional area and thickness is greater than the second cross-sectional area and is greater than the third cross-sectional area and thickness such that the plate projects in a fusiform manner both toward and away from the intervertebral disc space.

The plate can be at least partially made of a radiolucent material. The plate can be at least partially made of an implantable polymer. A first plate aperture of the at least one plate aperture can be asymmetric. A first bone screw of the at least one bone screw can be sized and shaped to be advanced along an insertional axis through the first plate aperture. Advancement of the first bone screw can result in a generally perpendicular translation of the plate relative to the insertional axis. A first bone screw of the at least one bone screw can be captured by a superimposition of a second bone screw of the at least one bone screw. The first bone screw can be immediately adjacent the second bone screw. The at least one bone screw can be secured to the plate with a locking mechanism. The at least one bone screw can include a shaft having a threaded region, a proximal head coupled to the shaft, and a threadless segment located distal to the proximal head and proximal to the threaded region. The locking mechanism can include a female thread form within the at least one plate aperture configured to engage the threaded region of the shaft and retain the at least one bone screw within the at least one plate aperture. The locking mechanism can include a tapered conical feature within the at least one plate aperture; and a shell having a generally cylindrical internal bore configured to be positioned coaxially around the threadless segment and a tapered conical external surface sized to form an interference fit with the tapered conical feature. The threadless segment can have a length being equal to or longer than a thickness of the at least one plate aperture through which the at least one bone screw is advanced and a diameter that is less than a major diameter of the threaded region of the shaft. The locking mechanism can include a deformable material forming at least a portion of the at least one aperture that is smaller in diameter than a major diameter of the threaded region of the shaft. Upon rotationally advancing the at least one bone screw through the at least one plate aperture, the threaded region can engage and deform the deformable material until a proximal extent of the threaded region is retained by the deformable material preventing reverse migration of the screw out of the aperture. The deformable material can be an implantable polymer.

In an interrelated aspect, described is a system for stabilizing adjacent superior and inferior vertebrae separated by an intervertebral disc space that includes a plate having at least one plate aperture and at least one bone screw sized and shaped to be positioned through the at least one plate aperture. The plate includes a first margin projecting from the plate and configured to contact the superior vertebra and a second margin projecting from the plate and configured to contact the inferior vertebra. The first and second margins projecting from the plate are configured to asymmetrically compress the intervertebral disc space. Prior to deployment a surface of the plate can be generally more concave than surface features of the adjacent superior and inferior vertebrae onto which the plate is being deployed.

In an interrelated aspect, described is a system for stabilizing adjacent superior and inferior vertebrae separated by an intervertebral disc space including a plate having at least one plate aperture and at least two pairs of projecting elements positioned on a surface of the plate configured to project toward the intervertebral disc space upon deployment of the system on the adjacent vertebrae; and at least one bone screw sized and shaped to be positioned through the at least one plate aperture. The at least two pairs of projecting elements are tapered and serve to align or fix the plate relative to the intervertebral disc space upon deployment of the system.

In an interrelated aspect, described is a system for stabilizing adjacent superior and inferior vertebrae separated by an intervertebral disc space including a plate having at least two plate apertures; a first bone screw sized and shaped to be positioned through a first of the at least two plate apertures along a first insertion axis above the intervertebral disc space; and a second bone screw sized and shaped to be positioned through a second of the at least two plate apertures along a second insertion axis below the intervertebral disc space. The first and second insertion axes of the first and second bone screws above and below the intervertebral disc space are convergent on a point in space. The point can be at least greater than a distance between a midpoint of the first bone screw and the second bone screw. The distance can be less than 50 cm.

### BRIEF DESCRIPTION OF THE DRAWINGS

Reference is made to the following description, taken in conjunction with the accompanying drawings, in which like reference characters refer to like parts throughout.

FIG. 1 is a perspective view of an implementation of a plate system;

FIG. 2 is a cross-sectional view of the plate system of FIG. 1;

FIG. 3 is a perspective view of a plate system incorporating an implementation of a dynamic compression mechanism;

FIG. 4 is a cross-sectional view of the plate system of FIG. 3 showing travel of a screw from a first position to a second position;

FIG. 5 is a side view of the plate system of FIG. 3 showing travel of a screw from a first position to a second position;

FIG. 6 is a partial cross-sectional view of a plate system incorporating an implementation of a plate locking mechanism;

FIG. 7A is a cross-sectional view of a plate system incorporating another implementation of a plate locking mechanism;

FIG. 7B is a side view of a bone screw from the plate system of FIG. 7A;

FIGS. 7C-7E are perspective views of portions of the plate locking mechanism of the plate system of FIG. 7A;

FIG. 8 is a partial cross-sectional view of a plate system incorporating another implementation of a plate locking mechanism;

FIG. 9 is a partial top plan view of an implementation of a plate system;

FIG. 10 is a partial perspective view of an implementation of a plate system;

FIG. 11 is a perspective view showing the convergence of screw insertion axes above and below the intervening disc space of an implementation of a plate system;

FIG. 12 is a cross-sectional view of an implementation of a plate system deployed on a pair of adjacent vertebrae;

FIG. 13 is a perspective view of a deep surface of an implementation of a plate system.

### DETAILED DESCRIPTION

Disclosed are intervertebral plate systems configured to be deployed in a patient adjacent the patient's spine. The plate systems described herein can be generally deployed in the spine using lateral and anterior approaches. In some implementations, lateral approaches can be used to access the lumbar and thoracic spine and anterior approaches can be used to access the cervical, thoracic and lumbar spine.

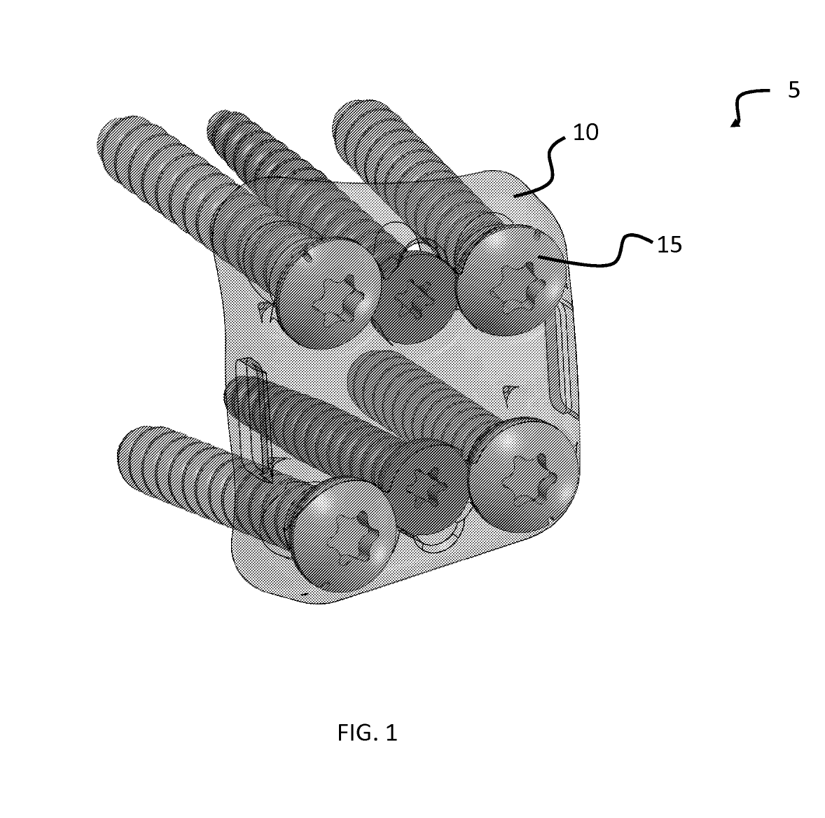

FIG. 1 is a perspective view of an implementation of a plate system 5. The plate system 5 can include a generally planar plate 10 having one or more apertures 20 through which one or more bone screws 15 can extend. The one or more bone screws 15 upon extending through the apertures can penetrate a portion of bone positioned under a deep surface of the plate to retain the plate 10. Generally, the plate system 5 described herein can be deployed in the spine and fixed to a portion or portions of the vertebral column. For example, the plate system 5 can be fixed to first and second adjacent vertebrae having an intervertebral disc space therebetween. In some implementations, the plate 10 can be positioned such that one or more bone screws 15 extending through the plate 10 from a superficial surface 25 to a deep surface 30 penetrate a portion of a superior vertebral body and one or more bone screws 15 extending through the plate 10 from the superficial surface 25 to the deep surface 30 penetrate a portion of an adjacent, inferior vertebral body such that the intervertebral disc space between adjacent vertebrae is at least partially covered by the deep surface 30 of the plate 10.

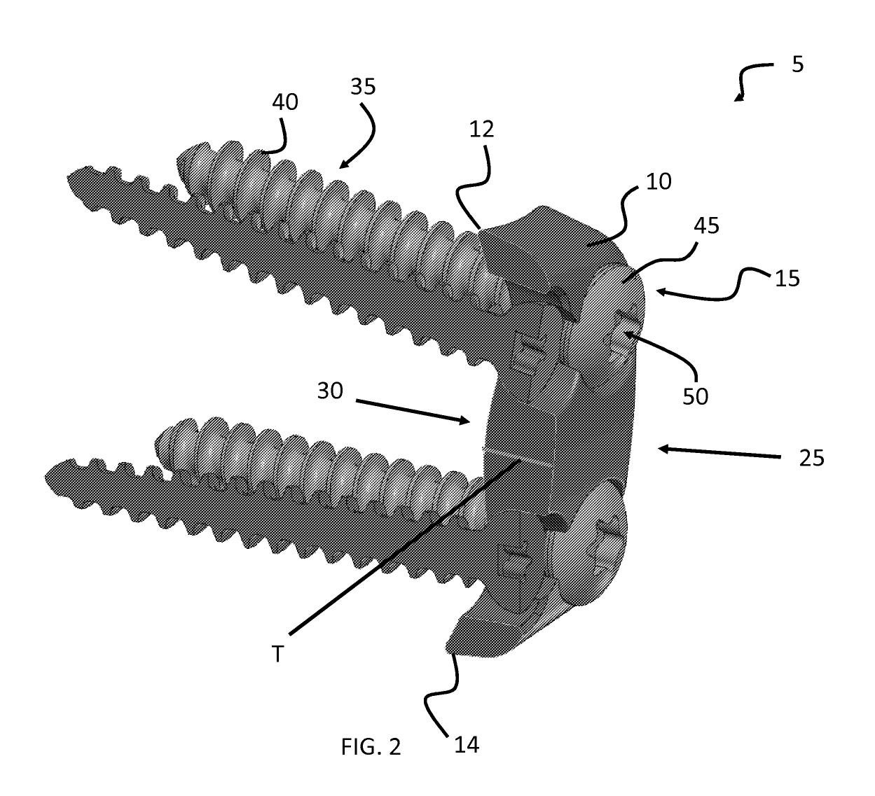

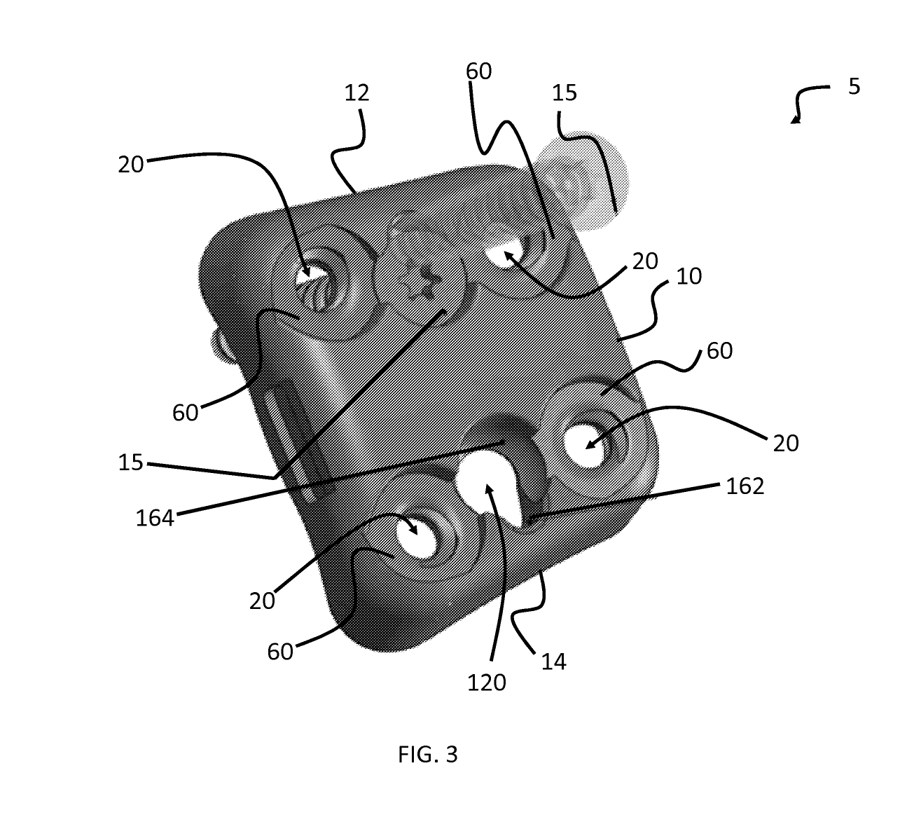

FIG. 2 is a cross-sectional view of the plate system 5 of FIG. 1. As mentioned above, the plate 10 can have a superficial surface 25 that upon deployment in the spine is configured to face outward away from the vertebrae to which the plate 10 is fixed and a deep surface 30 that is configured to face toward the vertebrae to which the plate 10 is fixed. The plate 10 can have a first cross-sectional area and thickness T from the superficial surface 25 to the deep surface 30 in a region near the center or midline of the plate 10. The plate 10 can taper towards one or both of the superior margin 12 (i.e. cephalad region) and inferior margin 14 (i.e. caudal region) of the plate 10. In turn, the margins 12, 14 can have a reduced thickness compared to the increased thickness of the plate 10 near the midline providing the plate 10 with a fusiform shape. In some implementations, the deep surface 30 can project both toward and away from the disc space upon deployment of the device in the spine. As best shown in FIG. 3, a first set of one or more apertures 20 can extend through the plate 10 near the superior margin 12 and a second set of one or more apertures 20 can extend through the plate 10 near the inferior margin 14. Upon implantation of the plate system 5 on the spinal column, the thicker central region of the plate 10 can be aligned with the intervertebral disc space and the thinner, margins 12, 14 of the plate 10 can be aligned with the adjacent superior and inferior vertebrae, respectively, such that the bone screws extending through the one or more apertures 20 can penetrate the underlying bone.

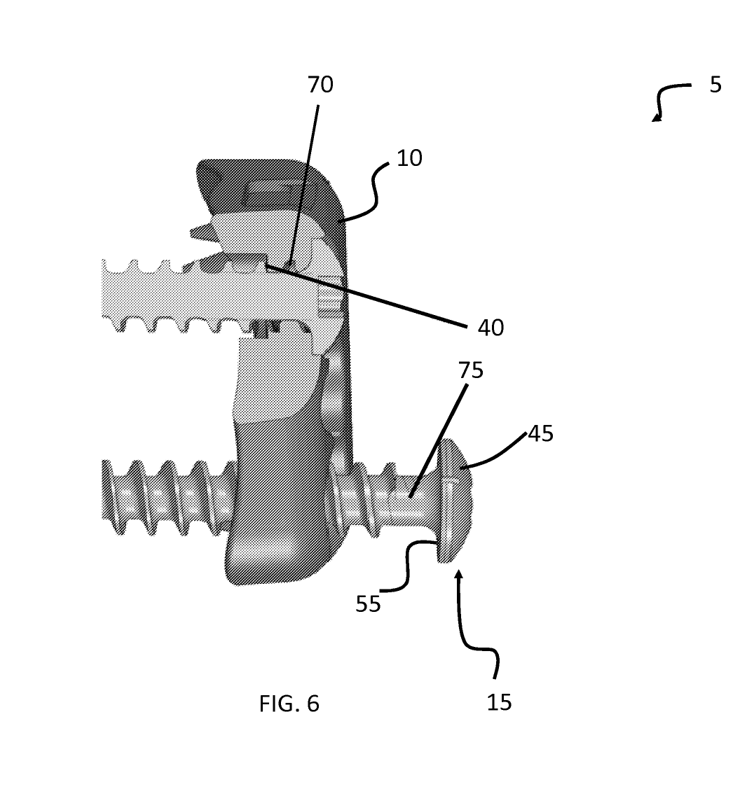

Again with respect to FIG. 2, each bone screw 15 can have a shank 35 on a leading end of the screw 15 having a minor diameter and a major diameter that are sized for extension through the aperture 20. The shank 35 can have an external thread 40 wrapped around the shank 35 for penetrating and fixing with bone that creates the major diameter of the shank 35. The screw 15 can also have a head 45 on a trailing end of the screw 15 that can have a surface feature 50 on an outer side of the head 45 that is configured to mate with a driving tool. The head 45 can have a larger diameter than the shank 35 such that a lower surface 55 of the head 45 (best shown in FIG. 6) abuts a bearing surface 60 surrounding the aperture 20 (best shown in FIG. 3) and prevents the screw 15 from being inserted completely through the aperture 20 of the plate 10. It should be appreciated that a variety of fasteners can be used with the plate system 5 described herein.

The plate systems described herein can accommodate and compress intervertebral implants and/or bone grafts positioned within the disc space between the adjacent vertebrae to be fused. FIGS. 3, 4, and 5 show a plate system incorporating an implementation of a dynamic compression mechanism. The plate system 5 can include a pair of compression apertures 120 through which a screws 15 can be advanced. The compression apertures 120 can be asymmetric and surrounded by an outer bearing surface 162 and an inner bearing surface 164. The outer bearing surface 162 of the compression aperture 120 can allow for the bone screw 15 to be initially inserted in a position that is away from the midline of the plate 10 and more towards the margins 12, 14. As the screw 15 is advanced further through the compression aperture 120, the lower surface 55 of the screw head 45 can abut the outer bearing surface 162 of the compression aperture 120 and be urged towards the inner bearing surface 164 of the compression aperture 120. This can cause the screw 15 to translate the bone through which it extends towards the midline of the plate 10 (i.e. the intervertebral disc space).

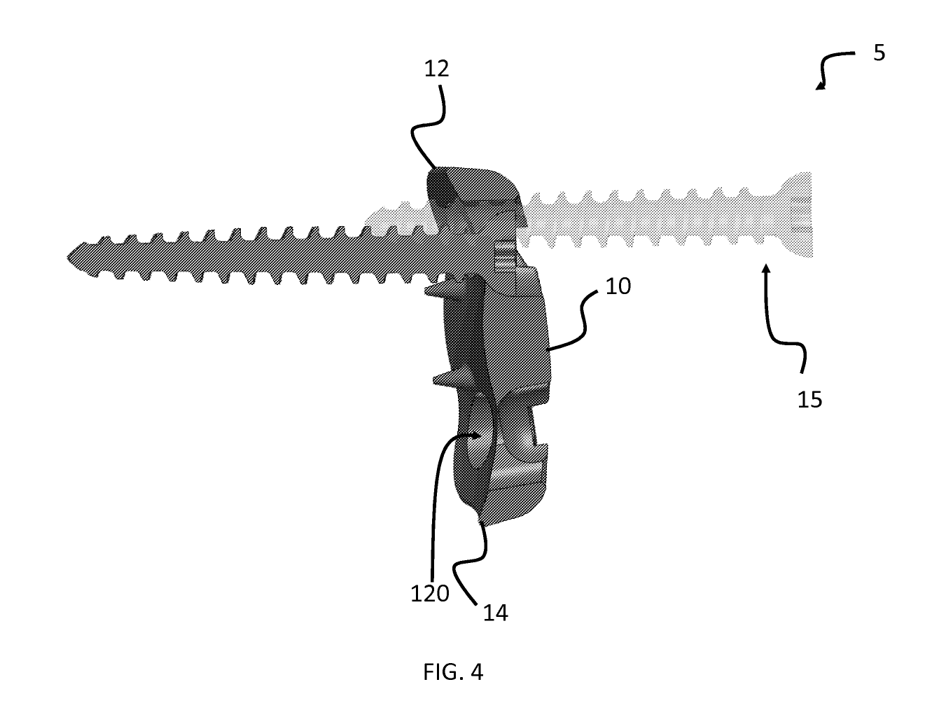

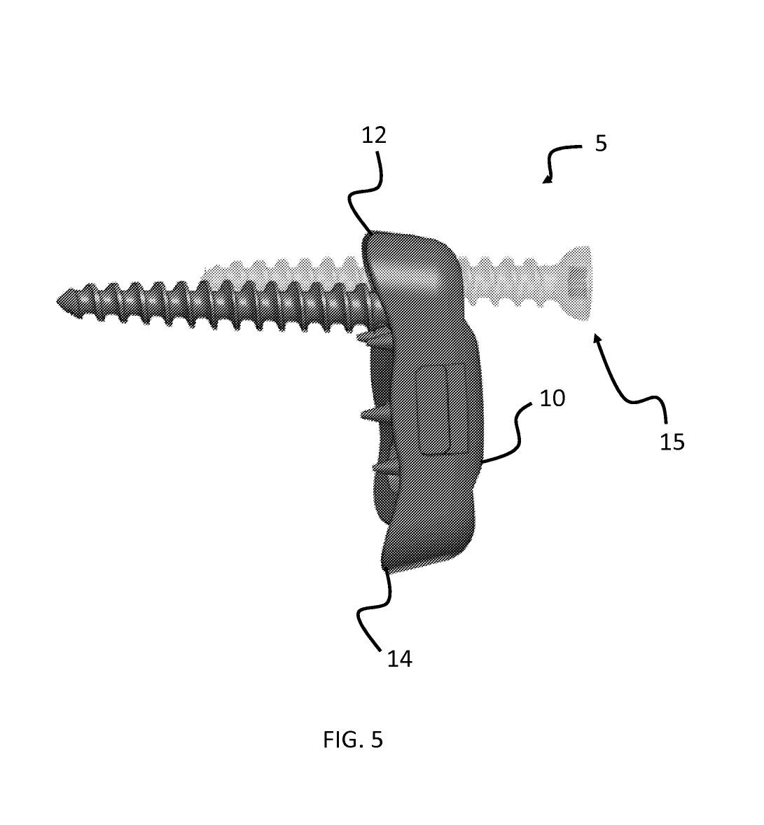

FIGS. 4 and 5 show a screw 15 being inserted through a compression aperture 120 near the superior margin 12 of the plate 10. The first position of the screw 15 prior to advancement can be located more superiorly than the second position after advancement of the screw 15. Advancement of the screw 15 through the compression aperture 120 into the superior vertebra urges the superior vertebra in a caudal direction towards the intervertebral disc space. A screw 15 can also be inserted through a compression aperture 120 located near the inferior margin 14 of the plate 10. The first position of the screw 15 prior to advancement can be located more inferiorly than the second position of the screw 15 after advancement. Thus, advancement of the screw 15 through the compression aperture 120 near the inferior margin 14 of the plate into the inferior vertebra can urge the inferior vertebra in a cephalad direction towards the intervertebral disc space. This configuration can result in a shorter distance (compared to the position that existed prior to dynamic compression plate screw advancement) between the two adjacent vertebrae immobilized by the plate 10 and associated screws 15.

FIG. 6 is a partial cross-sectional view of a plate system incorporating an implementation of a plate locking mechanism. At least one of the apertures 20 in the plate 10 can include a locking thread 70 that is configured to mate with the thread 40 of the bone screw 15. The locking thread 70 can be a female thread and the thread 40 of the bone screw 15 can be a male thread or vice versa. The female thread 70 can serve to obstruct the screw 15 from translating back through the aperture 20. The screw 15 can also include a reduction or discontinuity in the thread profile forming threadless segment 75 located below the screw head 45 that can act as a lag screw to compress the plate 10 against the bone.

It should be appreciated that other locking mechanisms between the plate and the screw are considered herein. For example, a plate screw interface is considered in which a collapsible bushing is used under the screw head. The collapsible bushing can have a truncated taper lock geometry externally and a slip fit, cylindrical geometry internally such that advancing the distal aspect of the screw head against the upper or proximal portion of the collapsible bushing can result in the bushing being driven within a mating truncated conical locking feature on the plate. In other implementations, the locking mechanism incorporated an unthreaded aperture formed of a compliant deformable material that provides an interference fit with the threadform of the screw upon advancement of the screw through the aperture.

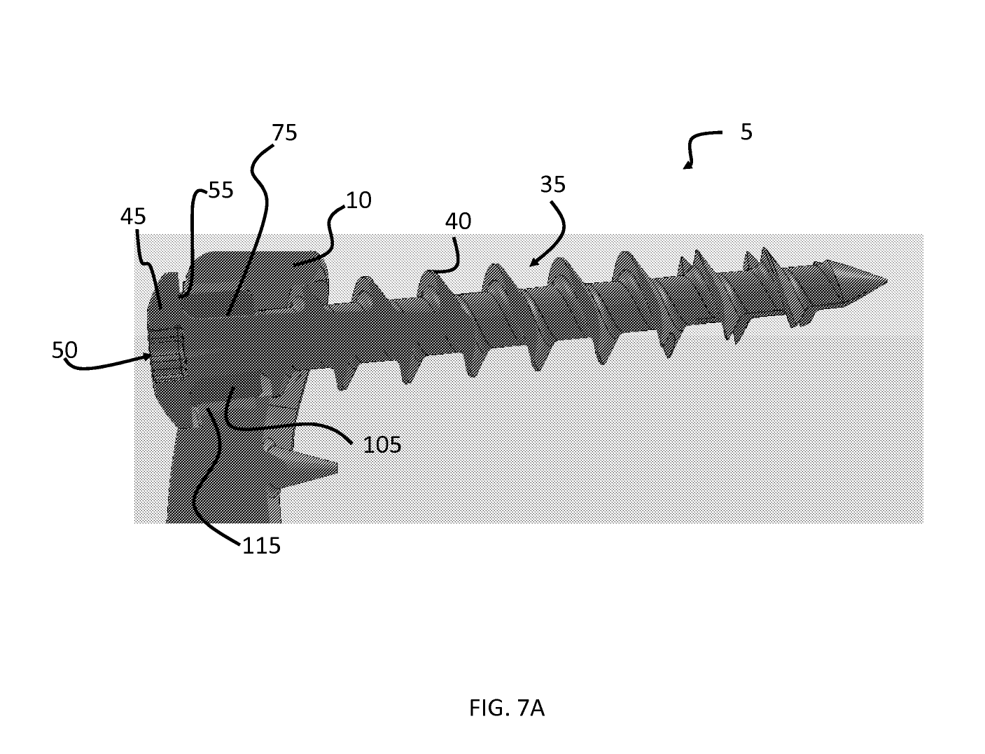

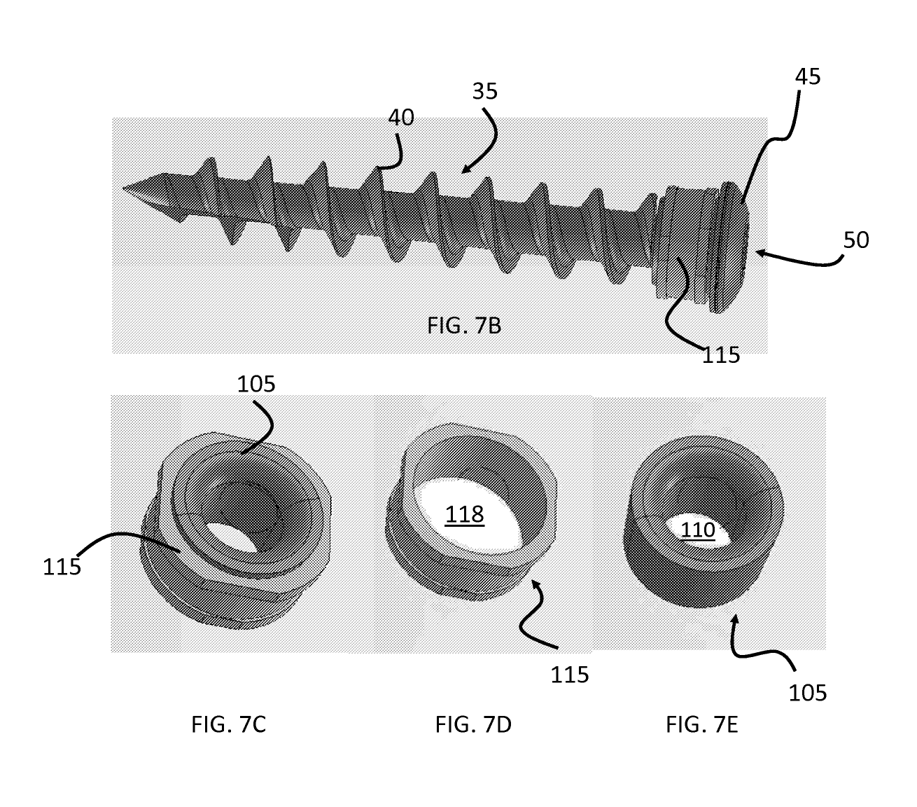

FIGS. 7A-7E illustrate a plate system incorporating another implementation of a locking mechanism. Instead of a threaded engagement between the screw and the plate, the locking mechanism incorporates the concept of a Morse taper to lock the screw 15 to the plate 10. A “snap on” feature can be incorporated below the screw head that can have a cylindrical internal bore and a locking conical Morse taper outer geometry that can mate-lock with a complimentary conical geometry within the aperture. As mentioned previously, the screw 15 can have a thread 40 that extends from a distal tip of the shaft 35 towards the head 45 and terminates on the shaft 35 a distance below the lower surface 55 of the head 45. The length of this threadless segment 75 can be equal to or longer than a thickness of the aperture 20 through which the screw 15 is advanced. The threadless segment 75 of the shaft 35 can have a diameter that is less than the major diameter of the threaded portion of the shaft 35.

A shell 105 (or pair of shells) can surround a length of the threadless segment 75 such that the shell 105 is positioned coaxially with the threadless segment 75 of the screw 15. The shell 105 can be a rigid element having a bore 110 extending from a proximal extent to a distal extent of the shell 105. The bore 110 can be generally cylindrical. The shell 105 can have a distal diameter that is less than the major diameter of the proximal extent of the threaded region of the screw 15. The external geometry of the surrounding shell 105 can be generally conical and associated with tapered lock dimensions, for example, an angle between two and six degrees (e.g. Morse taper) relative to the longitudinal axis of the shell 105.

A collar 115 can be fixed within an aperture 20 of the plate 10 such that the screw 15 and shell 105 can be advanced through the collar 115. The collar 115 can include an internal bore 118 and be formed of a rigid material. The bore 118 can be conical and tapered such that the bore 118 corresponds with the external taper lock geometry of the shell 105. Linear advancement of the shell 105 within the bore 118 of the collar 115 can result in a friction lock between the shell 105 and the bore 118 of the collar 115. It should be appreciated that the plate 10 may not incorporate a collar 115 and the friction lock can occur between the shell 105 and the aperture 20 of the plate 10. When the shaft 35 of the screw 15 is advanced into the vertebral bone, the shell 105 surrounding the threadless segment 75 of the screw 15 can advance within the rigid taper lock of the plate 10 resulting in a friction lock between the plate 10 and the surrounding shell 105. The friction lock can retain the screw 15 while permitting the screw 15 to be freely rotated relative to both the surrounding shell 105 and the plate 10. The external geometry of the collar 115 can provide a way for securing the collar 115, which can be a rigid component, to the plate 10, which can be an injection molded body. For example, the collar 115 can have upper and lower flanges that can prevent the migration of the collar 115 relative to the plate 10. The external or outer surface of one or both of the flanges can have a surface geometry, such as a flat, splined, knurled or other surface feature that can prevent the rotation of the collar 115 about its generally cylindrical axis with respect to the plate 10.

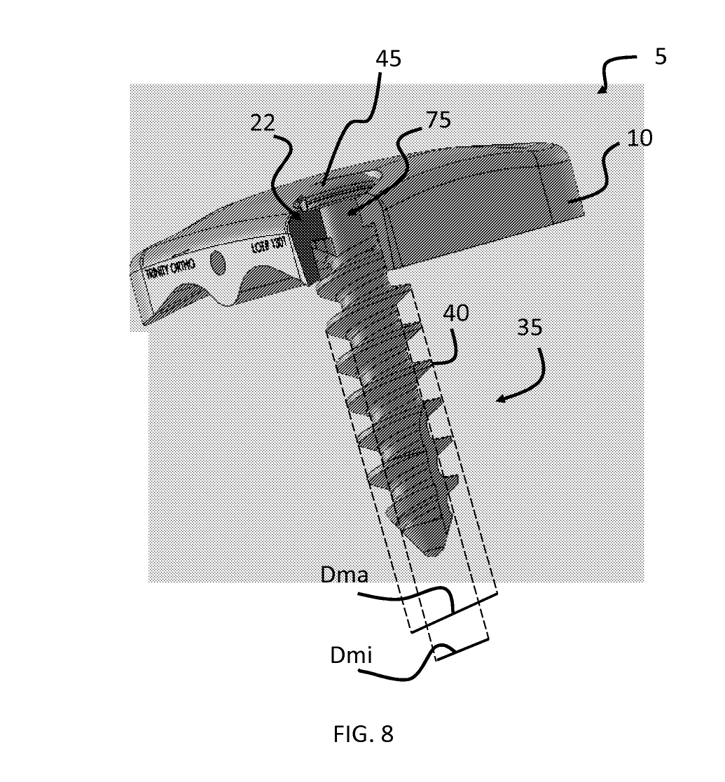

FIG. 8 illustrates a plate system incorporating another implementation of a locking mechanism that incorporates an interference fit between the thread of the screw and a deformable and/or compliant material in the aperture. The plate 10 can have an unthreaded aperture 20 extending through it that is sized equal to the minor diameter Dmi of the screw. As described above, the screw 15 can have a thread 40 that extends from the distal tip of the shaft 35 proximally, terminating a distance below the head 45 forming a threadless region 75. The length of the threadless region 75 can be equal to or longer than the thickness of the aperture 20 extending through the plate 10 through which the screw 15 is to be advanced. The threadless region 75 of the screw 15 can have a diameter that is less than the major diameter Dma of the threaded segment of the shaft 35. The aperture 20 of the plate 10 can be generally cylindrical and smaller in diameter than the major diameter Dma of the threaded segment of the screw shaft 35. At least a portion of the aperture 20 can include a deformable material 22. The diameter of the deformable material 22 of the aperture 20 can be less than the major diameter Dma of the thread 40. As the screw 15 is rotationally advanced through the aperture 20, the threaded region of the shaft 35 can engage and deform the deformable material 22 of the aperture 20. Once the screw 15 is fully advanced through the plate 10, the proximal extent of the thread 40 on the shaft 35 which is also less than the major diameter Dma of the thread 40 can be retained by the deformable material 22 of the aperture 20 and serve as a stop to prevent reverse migration of the screw 15 out of the plate 10. This configuration also can allow for the screw 15 to be freely rotated relative to the plate 10. The deformable material 22 of the aperture 20 can vary including, but not limited to, for example, implant-grade implantable polymers including polyether ether ketone (i.e. PEEK) or other compliant materials.

One or more regions of the plate 10 in addition to the aperture 20 can be formed of a deformable material such as an implantable polymer. The deformable material of the plate 10 can be the same as or a different material as the deformable material 22 of the aperture 20. One or more regions of the plate 10 can also be formed of a radiolucent material. Polymers such as PEEK are radiolucent and can provide an advantage that they do not impede observation of the implantation site. For example, a plate system 5 formed at least partially of radiolucent materials like PEEK can allow for assessment of the progression of bone growth between vertebrae during the post-operative period, which is generally assessed with the use of X-ray observation, either routine or with computer assisted tomography or CAT scans. Metal plates are generally stiffer than the bones to which they are attached. The transfer of loads from one vertebra to another via the plate can be in part stress shielded by the relatively stiff intervening metal plate. Polymers have a modulus that is more compliant than most implanted metals. Comparable immobilization using polymeric materials such as PEEK can be achieved although the cross sectional area may be greater than metal implants.

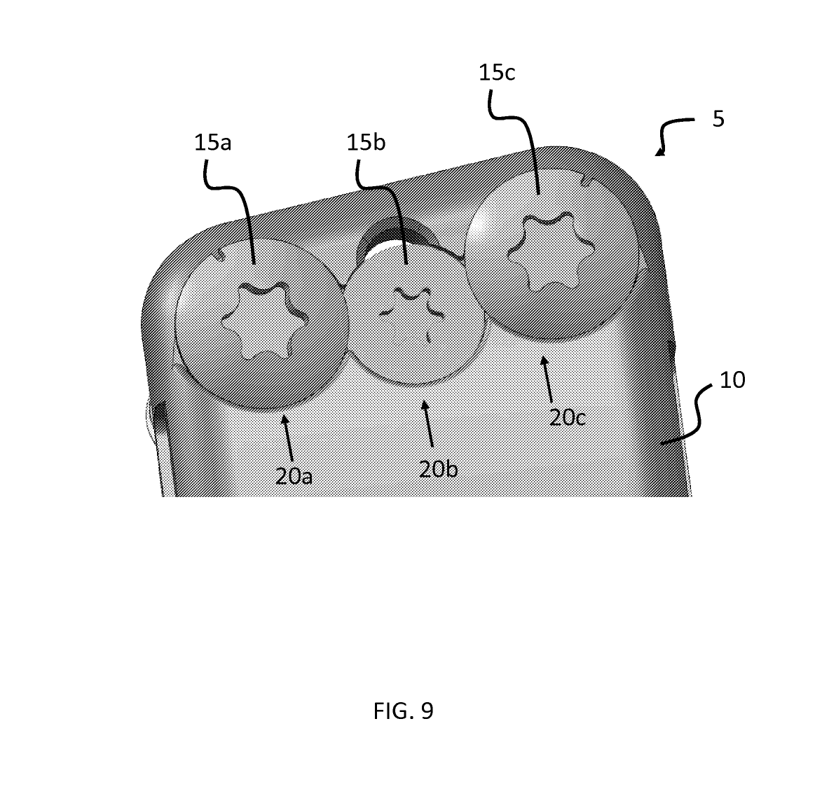

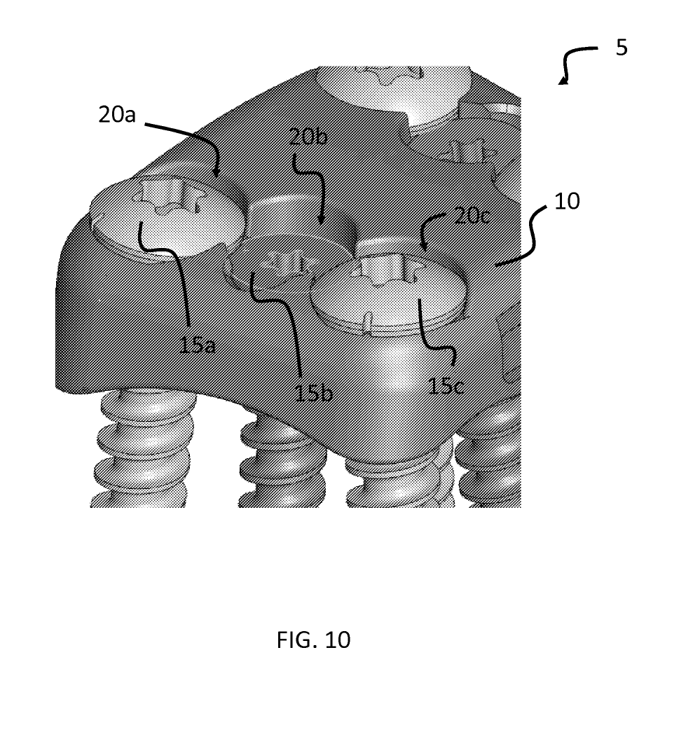

One or more of the screws inserted through the apertures in the plate can be captured by a superimposition of one or more of the other screws inserted through a different aperture in the plate. As shown in FIG. 9 and FIG. 10, the one or more apertures in the plate 10 can be positioned relative to one another such that the heads 45 of neighboring screws 15 or an immediately adjacent screws positioned therethrough interact with one another. For example, a first screw 15a can be inserted through a first aperture 20a and a second screw 15b can be inserted through a second aperture 20b, which can be a compression aperture. The head of the first screw 15a can interact with or contact the head of the second screw 15b in a manner that retains the second screw 15b within the plate 10 once both screws 15a, 15b are tightened. Further, a third screw 15c can be inserted through a third aperture 20c such that the head of the third screw 15c also interacts with or contacts the head of the second screw 15b such that the second screw 15b is additionally trapped within the plate 10 once all the screws 15a, 15b, 15c are tightened. The lower surface 55 of the heads 45 of the screws 15a, 15c can contact an upper surface of the head 45 of the screw 15b. Further, one or all of the apertures 20a, 20b, 20c can include locking threads 70 or another locking mechanism as described above such that tightening and locking one or both of the first and third screws 15a, 15c also locks the second screw 15b.

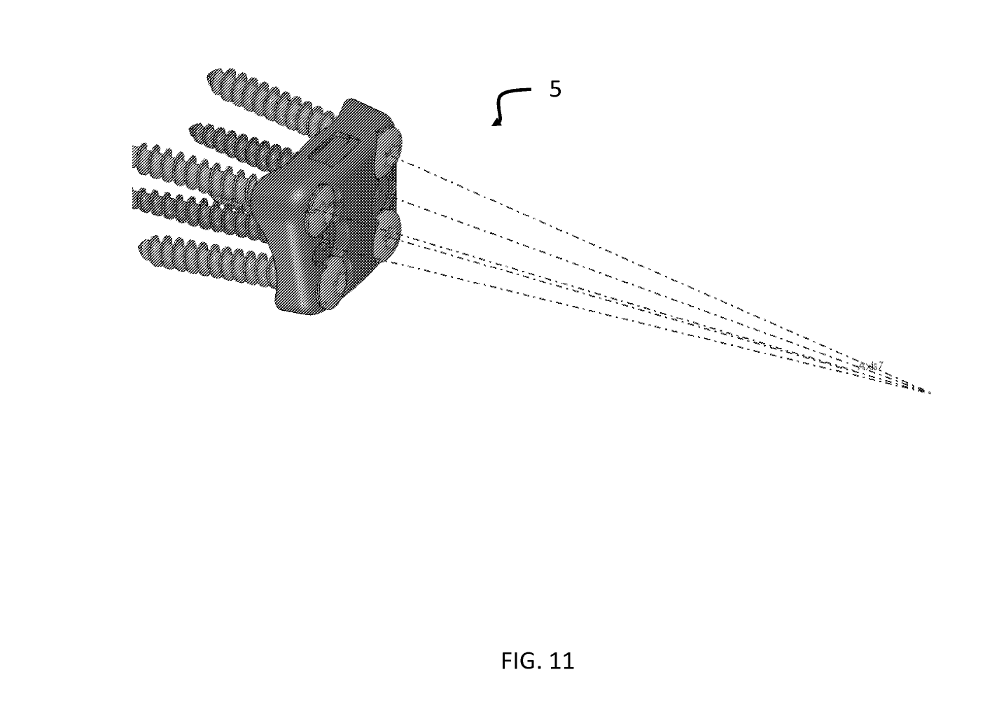

As described above, advancement of the screw can result in a generally perpendicular translation of the plate 10 relative to the insertional axis of the screw 10. A first bone screw 15 can be sized and shaped to be positioned through an aperture along a first insertion axis, for example above the intervertebral disc space. A second bone screw 15 can be sized and shaped to be positioned through another aperture along a second insertion axis, for example below the intervertebral disc space. The first and second insertion axes of the first and second bone screws above and below the intervertebral disc space can be convergent on a point in space. The point in space can be at least greater than a distance between a midpoint of the first bone screw and the second bone screw. The distance can be less than 50 centimeters (see FIG. 11). Designing the screw axis associated with the plate to converge at the point in space can facilitate the insertion of the screws through relatively narrow surgical approaches, thus reducing the demand of soft tissue dissection and retraction. This convergence of screw axes can provide for a reduced requirement for soft tissue retraction during screw pilot hole preparation, screw hole taping, and screw insertion.

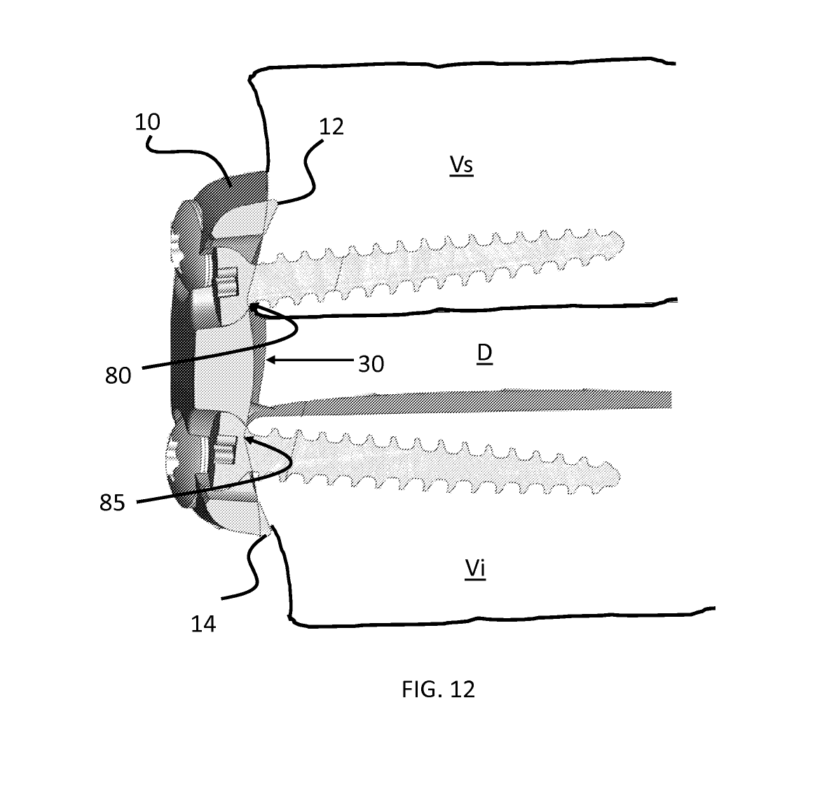

As shown in FIG. 12, the plate 10 can have a geometry on its deep surface 30 that includes a first concavity 80 and a second concavity 85. The first concavity 80 on the deep surface 30 of the plate 10 allows the superior margin 12 projecting from the plate 10 to contact the superior vertebra Vs near the cephalad extent of the superior vertebra Vs prior to the plate 10 contacting the superior vertebra Vs within the first concavity 80 near the caudal extent of the superior vertebra Vs. Similarly, the second concavity 85 on the deep surface 30 of the plate 10 allows the inferior margin 14 projecting from the plate 10 to contact the inferior vertebra Vi near its caudal extent prior to the plate 10 contacting the inferior vertebra Vi within the second concavity 85 near its cephalad extent. These deep surface concavities 80, 85 provide further angled dynamic compression of the adjacent vertebrae towards the contents of the intervertebral disc space D separately or in combination with compression provided by screw advancement through the compression apertures 120.

Prior to deployment, the deep surface 30 of the plate 10 can be generally more concave than surface features of the adjacent vertebrae to which the plate 10 is to be deployed that align with the plate 10. The plate can initially confer an increased convergence of the vertebrae to which the plate is affixed on the side of the disc space away from the side on which the plate is positioned, relative to the side to which the plate is immediately located. With additional screw advancement, concurrent plate compression and resistive loading of an intervertebral disc space implant, the plate 10 can bend or warp. The bending or warping of the plate 10 can lessen the concavity toward the disc space and result in “dynamization” of the plate 10. This can enhance stability and reduce the inclination for distraction of the opposite side of the disc space D as might otherwise occur with ipsilateral plate compression. This provides an asymmetric “angled” or “dynamic” compression of the intervertebral disc space, particularly when provided in conjunction with an intervening intervertebral device such as a cage or a stent. By compressing the disc space initially and preferentially on the side opposite the plate's affixed position, the resistance afforded by the intervertebral cage, spacer, stent, implant or other device that may be generally non-compressible can cause the plate to bow or “dynamize.”

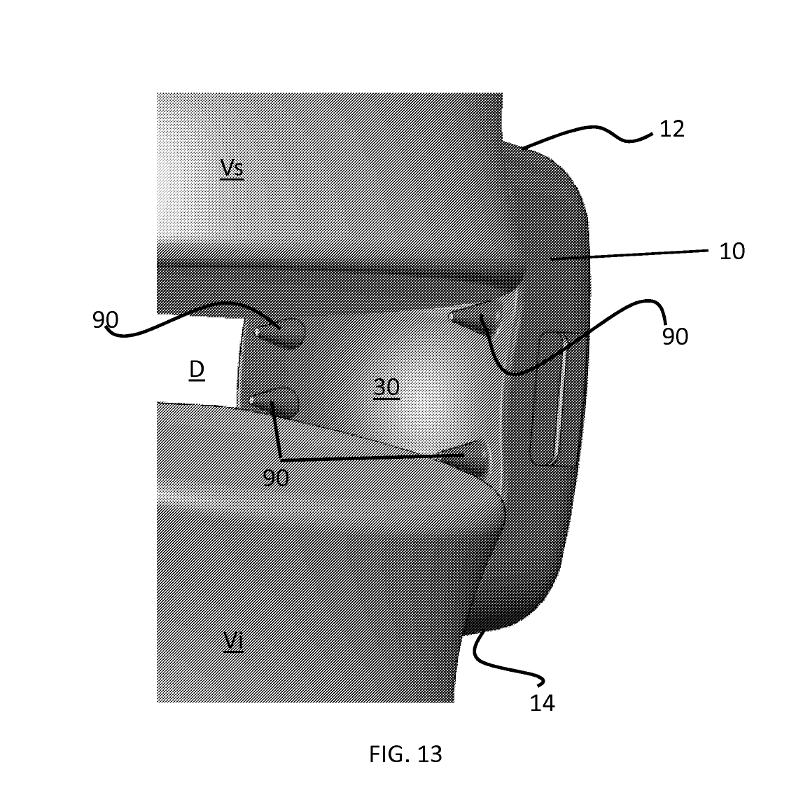

Now with respect to FIG. 13, the deep surface 30 of the plate 10 can also include one or more projecting features or projecting elements 90. The projecting elements 90 can be generally cylindrical or conical features that can taper towards a pointed tip on their distal-most extents. The plate 10 can include a pair of projecting elements 90 that are located closer to the inferior margin 14 of the plate 10 and a second pair of projecting elements 90 that are located closer to the superior margin 12 of the plate 10. The projecting elements 90 can provide for localized positioning of the plate 10 relative to the adjacent endplates of the superior vertebra Vs and the inferior vertebra Vi and intervening disc space D. A first set of projecting elements 90 can contact the superior vertebra Vs and a second set of projecting elements 90 can contact the inferior vertebra Vi. These projecting elements 90 can also provide for temporary stabilization of the plate 10, such as by piercing tissue or wedging between adjacent vertebrae.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. It should also be appreciated that sizes, materials, surface patterns and finishes can be altered to suit uses including extreme environments and loading to achieve required performance in those situations.

Although embodiments of various methods, systems and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system for stabilizing adjacent superior and inferior vertebrae separated by an intervertebral disc space, comprising:
a plate having at least one plate aperture, wherein the plate has a first cross-sectional area and thickness near a midline of the plate that is aligned with the intervertebral disc space upon deployment of the system, a second cross-sectional area and thickness located near a superior margin of the plate that is aligned with the superior vertebra upon deployment of the system, and a third cross-sectional area and thickness located near an inferior margin of the plate that is aligned with the inferior vertebra upon deployment of the system; and
at least one bone screw sized and shaped to be positioned through the at least one plate aperture,
wherein the first cross-sectional area and thickness is greater than the second cross-sectional area and is greater than the third cross-sectional area and thickness such that the plate projects in a fusiform manner both toward and away from the intervertebral disc space.

2. The system of claim 1, wherein the plate is at least partially made of a radiolucent material.

3. The system of claim 1, wherein the plate is at least partially made of an implantable polymer.

4. The system of claim 1, wherein a first plate aperture of the at least one plate aperture is asymmetric.

5. The system of claim 4, wherein a first bone screw of the at least one bone screw is sized and shaped to be advanced along an insertional axis through the first plate aperture.

6. The system of claim 5, wherein advancement of the first bone screw results in a generally perpendicular translation of the plate relative to the insertional axis.

7. The system of claim 1, wherein a first bone screw of the at least one bone screw is captured by a superimposition of a second bone screw of the at least one bone screw, wherein the first bone screw is immediately adjacent the second bone screw.

8. The system of claim 1, wherein the at least one bone screw is secured to the plate with a locking mechanism.

9. The system of claim 8, wherein the at least one bone screw comprises a shaft having a threaded region, a proximal head coupled to the shaft, and a threadless segment located distal to the proximal head and proximal to the threaded region.

10. The system of claim 9, wherein the locking mechanism comprises a female thread form within the at least one plate aperture configured to engage the threaded region of the shaft and retain the at least one bone screw within the at least one plate aperture.

11. The system of claim 9, wherein the locking mechanism comprises:
a tapered conical feature within the at least one plate aperture; and
a shell having a generally cylindrical internal bore configured to be positioned coaxially around the threadless segment and a tapered conical external surface sized to form an interference fit with the tapered conical feature.

12. The system of claim 11, wherein the threadless segment has a length being equal to or longer than a thickness of the at least one plate aperture through which the at least one bone screw is advanced and a diameter that is less than a major diameter of the threaded region of the shaft.

13. The system of claim 9, wherein the locking mechanism comprises a deformable material forming at least a portion of the at least one aperture that is smaller in diameter than a major diameter of the threaded region of the shaft,
wherein upon rotationally advancing the at least one bone screw through the at least one plate aperture, the threaded region engages and deforms the deformable material until a proximal extent of the threaded region is retained by the deformable material preventing reverse migration of the screw out of the aperture.

14. The system of claim 13, wherein the deformable material comprises an implantable polymer.

15. A system for stabilizing adjacent superior and inferior vertebrae separated by an intervertebral disc space, comprising:
a plate having at least one plate aperture and a first margin projecting from the plate and configured to contact the superior vertebra and a second margin projecting from the plate and configured to contact the inferior vertebra; and
at least one bone screw sized and shaped to be positioned through the at least one plate aperture,
wherein the first and second margins projecting from the plate are configured to asymmetrically compress the intervertebral disc space.

16. The system of claim 15, wherein prior to deployment a surface of the plate is generally more concave than surface features of the adjacent superior and inferior vertebrae onto which the plate is being deployed.

17. A system for stabilizing adjacent superior and inferior vertebrae separated by an intervertebral disc space, comprising:
a plate having at least one plate aperture and at least two pairs of projecting elements positioned on a surface of the plate configured to project toward the intervertebral disc space upon deployment of the system on the adjacent vertebrae; and
at least one bone screw sized and shaped to be positioned through the at least one plate aperture,
wherein the at least two pairs of projecting elements are tapered and serve to align or fix the plate relative to the intervertebral disc space upon deployment of the system.

18. A system for stabilizing adjacent superior and inferior vertebrae separated by an intervertebral disc space, comprising:
a plate having at least two plate apertures;
a first bone screw sized and shaped to be positioned through a first of the at least two plate apertures along a first insertion axis above the intervertebral disc space; and
a second bone screw sized and shaped to be positioned through a second of the at least two plate apertures along a second insertion axis below the intervertebral disc space,
wherein the first and second insertion axes of the first and second bone screws above and below the intervertebral disc space are convergent on a point in space.

19. The system of claim 18, wherein the point is at least greater than a distance between a midpoint of the first bone screw and the second bone screw.

20. The system of claim 17, wherein the distance is less than 50 cm.
